(19) United States
(12) Patent Application Publication
(10) Pub. No.: US 2004/0052761 A1
(43) Pub. Date: Mar. 18, 2004

(54) Localized delivery system for cancer drugs, phenstatin, using N-isopropylacrylamide

(76) Inventors: Brent Vernon, Mesa, AZ (US); Steven Powell, Vermillion, SD (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/624,294

(22) Filed: Jul. 21, 2003

Related U.S. Application Data
(60) Provisional application No. 60/397,182, filed on Jul. 19, 2002.

(51) Int. Cl. A61K 31/74

(52) U.S. Cl. 424/78.18

(57) ABSTRACT

An injectable drug delivery system for localized release of Phenstatin to a tumor site over a period of time is provided. The drug delivery system comprises the thermoresponsive polymer N-isopropylacrylamide (NIPAAM) and Phenstatin, a toxic antineoplastic agent. The drug delivery system has a critical solution temperature (LCST) that causes it to change from the liquid state at room temperature when injected to a gel or semi-solid state after reaching the temperature of the human body in situ.

Methods are given for delivering Phenstatin to a cancerous tumor. In these methods, the drug delivery system is injected into a tissue or into a tumor where it forms a gel. Phenstatin is slowly released from the polymer and exerts its cytotoxic, tublin-related effects on the tumor. Tumors that may be treated by the present methods include, but are not limited to breast, prostate, lung and bowel cancerous tumors.

### CLAIM TO DOMESTIC PRIORITY

This application claims priority to U.S. Provisional application Serial No. 60/397,182 entitled “Localized Delivery System for Cancer Drugs, Phenstatin, Using N-isopropylacrylamide” Jul. 19, 2002, by Brent Vernon et al., and is herein incorporated by reference in its entirety.

### FIELD OF THE INVENTION

This invention concerns delivery systems for antineoplastic agents and, more specifically, is directed to an injectable localized delivery system comprising Phenstatin and the thermoreversible hydrogel N-isopropylacrylamide (NIPAAm).

### BACKGROUND OF THE INVENTION

Intravenous delivery is generally used to deliver an anticancer drug to a tumor site. However, intravenous delivery often results in a high system concentration of the drug that can cause devastating side effects due to the destruction of healthy cells (3,4). Localized delivery systems have been sought that deliver an anticancer drug locally to the tumor to reduce the systemic levels of the drug, thus minimizing undesirable side effects.

Systems comprising thermoreversible hydrogels have been developed for localized delivery of anticancer drugs. See, e.g. U.S. Pat. No. 6,201,072 to Rathi et al., U.S. Pat. No. 6,193,991 to Shukla. One such thermoreversible hydrogel is N-isopropylacrylamide (NIPAAm) (5). NIPAAm has a low critical solution temperature (LCST) at 32° C. (6). Aqueous solutions of these polymers are soluble below their LCST but precipitate above their LCST. This property allows NIPAAm to be water-soluble at room temperature (25° C.) and insoluble at body temperature (37° C.). Other hydrogels with thermal sensitivities similar to NIPAAm are known, but NIPAAM's quick phase transition makes it a desirable candidate for injectable localized delivery systems.

Phenstatin is a cancer drug that is currently under preclinical development (1,2). It was derived from a class of antineoplastic drugs called the Combretastatins. These drugs were isolated from the African willow tree Combretum caffrum Kuntze (Combretaceae) (1). Phenstatin is a potent inhibitor of tubulin polymerization and the binding of colchicines to tubulin. (1) The tubulin inhibition stops the development of growing blood vessels and dividing cells. (See, e.g. U.S. Pat. No. 6,593,374 to Pinney et al. ) Phenstatin cuts off the blood supply to the growing tumor and essentially “starves” the tumor to death.

Methods have been sought to incorporate Phenstatin into NIPAAm to allow injectable localized delivery of Phenstatin into a tumor site and provide more effective methods of tumor treatment.

### BRIEF DESCRIPTION OF THE FIGURES

FIG. 1 shows Fourier transform infrared spectroscopy (FTIR) (for (1) acrylated phenstatin, (2) poly(N-isopropylacrylamide-co-phenstatin) and (3) poly(N-isopropylacrylamide) between 1200 and 1500 cm-1. Distinguishing peaks found in acrylated phenstatin but not in poly(N-isopropylacrylamide) at 1416 cm-1 and 1334 cm-1 were also found in the poly(N-isopropylacrylamide-co-Phenstatin).

FIG. 2 is a graph of the proton nuclear magnetic resonance (NMR) profile of acrylated phenstatin.

### SUMMARY

An injectable drug delivery system for localized release of a therapeutically effective amount of Phenstatin to a tumor site over a period of time is provided. The drug delivery system comprises the thermoresponsive polymer N-isopropylacrylamide (NIPAAM) and Phenstatin, a toxic antineoplastic agent. The drug delivery system has a low critical solution temperature (LCST) that causes it to change from the liquid state at room temperature, when injected, to a gel or semi-solid state after reaching the temperature of the human body. Phenstatin is released over a period of time from the implanted NIPAAm/Phenstatin.

The drug delivery system may be prepared by combining Phenstatin acrylate and NIPAAm under polymerization conditions. In certain preferred embodiments, Phenstatin acrylate is prepared by reacting Phenstatin with acryloyl chloride (ACL). In certain other preferred embodiments, Phenstatin acrylate is prepared by reacting Phenstatin with isopropenyl chloroformate (IPCF)

In certain preferred instances, the NIPAAm is copolymerized with acrylic acid (AAc) to maintain the LCST of the drug delivery system near body temperature. The incorporated drug increases the LCST of the polymer whereas the LCST decreases with concentration of drug.

In an important aspect of the present invention, methods are given for delivering Phenstatin to a cancerous tumor. In these methods, the drug delivery system is injected into a tissue or directly into the tumor where it forms a gel. Phenstatin is slowly released from the polymer and exerts its cytotoxic, tubulin-related effects on the tumor. Tumors that may be treated by the present methods include, but are not limited to breast, prostate, lung and bowel cancerous tumors.

### DETAILS

A thermoreversible drug delivery system for localized injection of cytotoxic drugs is provided. The delivery system is polymeric in nature and comprises N-isopropylacrylamide (NIPAAm), a thermoreversible polymeric hydrogel and Phenstatin, an anti-tumor agent.

At temperatures below its LCST, its gelation temperature, a thermoreversible hydrogel is a liquid, and at temperatures at or above the gelation temperature, the composition is a gel or semi-solid. The LCST of NIPAAm is 32° C. (6). This property causes NIPAAm to be in a liquid state and water-soluble at room temperature (25° C.) but insoluble at body temperature (37° C.). NIPAAm's quick phase transition at 32° C. makes it useful in an injectable delivery system in warm-blooded animals.

In the present drug delivery system, Phenstatin is acrylated and then polymerized with the NIPAAm. Because Phenstatin is a non-polar, hydrophobic drug, the LCST of the system decreases as more drug is added, since the non-polar groups on the drug reduce the polymer's solubility in water. This may be observed by DSC of a model system comprising Isovanillin, a structurally similar compound to Phenstatin. Table 1 shows the peak of the DSC thermogram for the model polymers with Isovanillin content of 0, 1, 2 and 5 mole per cent Isovanillin.

TABLE 1
LCST of NIPAAm/Isovanillin with 0, 1, 2 and 5 mol % Isovanillin

| SAMPLE (mol % ISV) | LCST (° C.) |
|---|---|
| 0 | 32 |
| 1 | 31.7 |
| 2 | 30.2 |
| 5 | 26.2 |

After the drug concentration is over 5 mol %, the LCST may not show linear properties and the LCST will decrease at a very large rate as more drug is added.

Accordingly, the concentration of Phenstatin in preferred embodiments of the present invention is chosen to provide an LCST that is between room temperature (25° C.) and body temperature (37° C.). Although higher drug concentrations are preferred to provide greater dosage in situ, the limits of concentration are constrained by the LCST at higher concentrations. In the NIPAAm/Phenstatin compositions of the present invention, the drug concentration is preferably about 5%. At higher concentrations, as seen in Table 1, the LCST will be too low.

In addition to affecting the LCST, the addition of drug to the polymer also affects the breadth of the phase transition. In the homopolymer NIPAAm, the phase transition from a liquid to a solid hydrogel is over a narrow temperature range. From the DSC data of the model polymers, it was observed that this phase change occurs over a much larger temperature range at higher concentrations of drug. The breadth of phase transition affects the therapeutic effectiveness of a composition. The broad phase change is due to the amount of drug on each individual polymer. If the drug is incorporated heterogeneously into the polymer, each chain may have a varying amount of drug, but the sample as a whole will have the same average value. Because there are varying amounts of drug on each chain, different chains will start their phase changes at different times. These varying phase changes cause the broadness of the peaks. A narrower transition range may be achieved by fractionation of the polymers to obtain a preferred polymer sample.

The release rate of the drug may be adjusted by changing various parameters such as hydrophobic/hydrophilic component content, polymer concentration, molecular weight and polydispersity of the polymer. Because the polymer is amphiphilic, it functions to increase the solubility and/or stability of drugs in the composition. The release rate of Phenstatin from the polymer may be increased by incorporation of a carbonate bond in the link between Phenstatin acrylate and the polymer. The carbonate bond is less stable than an ester bond and therefore offers greater release rate.

The preparation of Phenstatin is disclosed in Pettit, G. R. et al. Antineoplastic Agents: 443 Synthesis of the cancer cell growth inhibitor hydroxyphenstatin and its sodium diphosphate prodrug”; Journal of Medicinal Chemistry, 2000, 43(14); p. 2731-2737 and Pettit, G. R. et al. Antineoplastic agents 379 Synthesis of Phenstatin phosphate; Journal of Medicinal Chemistry, 1998 41 (10) p. 1688-1695 which are herein incorporated in their entirety.

The drug delivery system of the present invention is prepared by polymerizing Phenstatin acrylate with NIPAAm to make NIPAAm/Phenstatin.

In a preferred method for preparing the NIPAAm/Phenstatin, Phenstatin acrylate was prepared by combining Phenstatin and acryloyl chloride (ACL) in a suitable solvent. The Phenstatin acrylate is then polymerized with NIPAAM to make a polymer containing preferably about 5 mol % acrylate and 95 mol % NIPAAm. In this preparative method, ester bonds results.

In other preferred methods Phenstatin acrylate may be prepared by combining Phenstatin and Isopropenyl Chloroformate (IPCF) in a suitable solvent under conditions for reaction. The Phenstatin acrylate may then be polymerized with NIPAAm to make a polymer most preferably containing about 5 mol % acrylate and 95 mol % NIPAAm In this reaction scheme, a carbonate bond results. This bond is less stable than an ester bond and thus provides faster release of Phenstatin agent in situ.

Most preferably the polymerization of Phenstatin acrylate and NIPAAm comprises the copolymerization with acrylic acid AAc. With AAc, the temperature of the LCST of the drug delivery system increases. This effect overcomes the effect of the addition of the drug to the polymer that causes the LCST (gel temperature) to decrease. By adding the AAc group, the LCST will be raised again. Thus higher concentrations of drug, greater than 5%, preferably 5% to 10%, most preferably 5% or greater may be incorporated into the drug delivery system for more effective toxic action in situ.

In an important aspect of the present invention, methods are presented for delivering the anticancer drug Phenstatin to a tissue for destruction of a tumor therein. The drug delivery system may be administered to a warm-blooded animal as a liquid or in a biologically compatible solvent by parenteral, ocular, topical, inhalation, transdermal, vaginal, transurethral, rectal, nasal, oral, pulmonary or aural delivery means. The composition may also be administered as a gel. Preferably the drug is injected locally to a tumor in a tissue where is it released at a controlled rate from the gel at the site of delivery.

Tumors that may be treated by the present drug delivery system include, but are not limited to breast, prostate, lung and bowel tissue.

The following examples are offered by way of illustration and not by way of imitation.

### EXAMPLES

### Example 1

This example illustrates the preparation of NIPAAm-isovanillan, a structurally similar compound to Phenstatin.

Materials

N-isopropylacrylamide (Sigma-Aldrich) was purified by recrystallization in hexane (10 g/100 ml at 40° C. to room temperature). Anhydrous dichloromethand, a,a-azoisobutylronitrile (AlBN), Isovanillin, acryloyl chloride, triethylamine, tetrahydrofuran (THF) hydrochloride acid (HCl) and hexane were obtained from Sigma-Aldrich. AlBN was purified by recrystallization in methanol (1 g/10 ml), dissolved at room temperature and recrystallized at −20° C.). Other materials were used as received.

Acrylation of Isovanillin

An acrylic group was added to Isovanillin by reacting Isovanillin with acryloyl chloride.

Five grams of Isovanillin was added to 100 mL of anhydrous dichloromethane (DCM). While stirring, 10 mL of triethylamine was added to the Isovanillin/DCM. The resulting mixture was stirred until it completely dissolved. After placing the Isovanillin on ice, 5 mL of acryloyl chloride was added to 5 mL of anhydrous DCM and added to the Isovanillin dropwise. The reaction was allowed to stand 4 hours under a N₂ atmosphere.

The reaction mixture was then taken off the ice bath and filtered. Four passes of 1N HCl, volume of 100 mL each, were then used to extract the remaining triethylamine in the reaction. The reaction mixture was then dropped into 900 mL of hexane, heated at 40° C. This was then filtered by vacuum filtration. Once the hexane was filtered, its volume was then reduced with a rotary evaporator. When the volume was reduced to 200 mL, the mixture was allowed to recrystallize. The product was then collected by vacuum filtration.

Polymerization of Isovanillin Acrylate (5 mol % Acrylate)

After developing the acrylate, it was then ready to be polymerized with NIPAAm.

Polymers with mole ratios of 00.1, 98:2 and 95:5 NIPAAm to acrylate were prepared using free radical polymerization. The monomers were combined in THF at 10 wt % with 7×10−3 mols of AlBN as initiator per mol of monomer. The polymerization occurred at 60° C. under a N₂ atmosphere, in the dark, overnight. The product was collected by precipitation in diethyl ether and vacuum filtered.

DSC Analysis of Polymers

The LCST data (table 1) was acquired from differential scanning calorimeter (DSC) (CSC4100 multi-cell differential scanning calorimeter; Calorimetry Science Corp., American Fork, UTAA) at a heating rate of 1° C./minute and over a range of −10 to 70° C. A phosphate buffered saline (PBS) solution with pH 7.4 was used as a baseline. Polymers were dissolved at 1 wt % in the same PBS buffer.

NMR

1H NMR spectra were recorded at 500 MHz using a Varian Inova 500 spectrometer. Samples were dissolved in CDCl3 and spectra were obtained at 25° C. Resonance assignments of precursors and polymers were confirmed as necessary using 2-dimensional gradient COSY, HMOC and HMBC spectra.

### Example 2

This example illustrates co-polymerizatin of NIPAAm and Acrylic Acid in the preparation of the drug delivery system.

In this Example Phenstatin acrylate was prepared as given in Example 3 or Example 4. However, in the polymerization step the NIPAAm is copolymerized with acrylic acid AAc. By using AAc, the temperature that the polymer will gel at (become insoluble), will increase. This effect is desirable because the addition of the drug to the polymer will cause the LCST (gel temperature) to decrease. By adding the AAc group, the LCST will be raised again.

The preparation of 5 mL of Tetrahydrofuran (THF) is added to the contents of RBF from Example 3 or Example 4. Next, 100 mg of acrylate, 104 mg of AlBN and 0.53 g of NIPAAm are added to the THF. The mixture is then allowed to polymerize at 60° C. for 4 hours while stirring. The reaction is covered with aluminum foil during polymerization to reduce light. Once the reaction is complete, it is dropped into 50 mL of diethyl ether. The precipitate is then collected by vacuum filtration. The polymer is further dried in a desiccator under vacuum.

### Example 3

This example illustrates the preparation of N-isopropylacrylamide (NIPAAm-phenstatin)with an ester bond.

The reaction scheme is as follows:

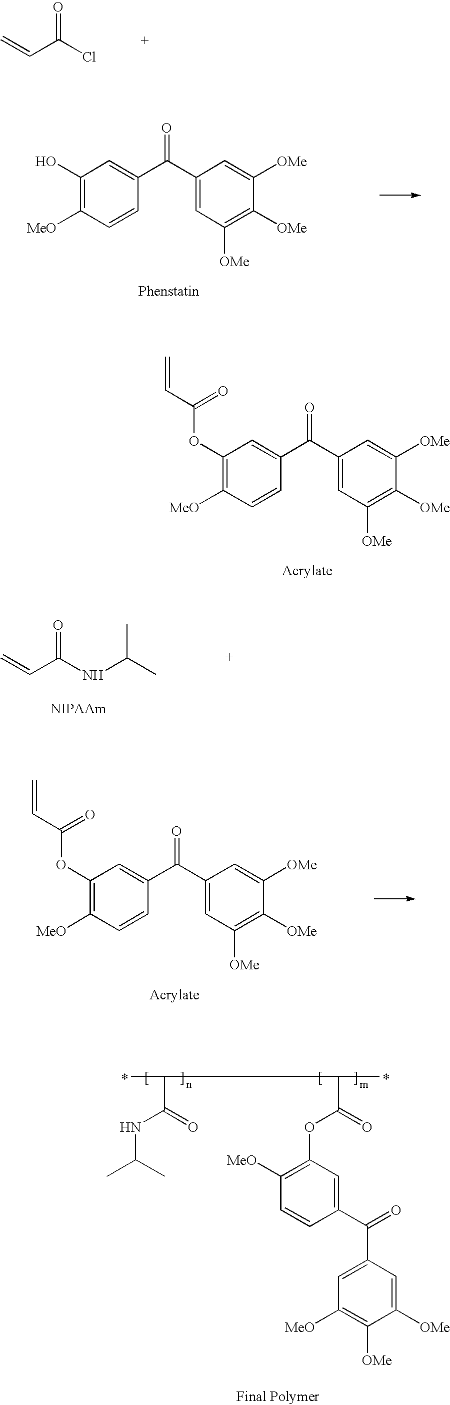

An acrylic group was added to Phenstatin by reacting Phenstatin with acryloyl chloride

Acrylation of Phenstatin (Ester Linkage)

Before starting the acrylation, all glassware (Round Bottom Flask (RBF), Dropper Funnel, Glass funnel, graduated cylinder) were dried in an oven overnight. Once the glassware was dried, it was then placed on a condenser and nitrogen gas was sent through the system as it cooled. This allowed for minimal condensation on the glassware as it cooled. After the glassware was set up and dried, 10 mL of anhydrous dichloromethane (DCM) was measured into the RBF. Next, 100 mg of Phenstatin was measured into the RBF. A stir bar was added to stir the mixture. After the Phenstatin was added, 98 μL of triethylamine was pipetted into the RBF. Once all the chemicals were added to the RBF, the mixture was stirred until it dissolved. When the mixture dissolved, it was then placed on an ice bath. The dropper funnel was then closed and 1 mL of DCM was added. After the DCM was added, 38 μL of Acryloyl Chloride (ACL) was carefully added to the DCM in the dropper funnel. The ACL and DCM were allowed to mix thoroughly. Once they were mixed, the DCM/ACL mixture was slowly dropped into the RBF over the ice bath. After all the solution was dropped into the RBF, the dropper funnel was closed and the reaction was allowed to proceed for 2-4 hours.

The acryl ate was then collected by taking the reaction off of the ice bath. The mixture was filtered by vacuum filtration to remove the triethylamine salt formed as a byproduct. Next, the reaction mixture was added to a separatory funnel. Four passes of 1 N HCl, volume of 10 mL each, were then used to extract the remaining triethylamine. The reaction mixture was then dropped into 90 mL of 50/50 hexane/ethyl acetate while being stirred. This was then filtered by vacuum filtration. Once the mixture had been filtered, its volume was reduced by using a rotary evaporator. The volume was reduced from 10 mL to about 5 mL. Once its volume was reduced to 5 mL, the hexane/ethyl acetate was placed on an ice bath so that the contents could recrystallize. The hexane/ethyl acetate was then filtered by vacuum filtration and the product was collected. The product was placed into a small tared scintillation vial and it was dried in a dessicator under vacuum.

Polymerization of Phenstatin Acrylate (5 mol % Acrylate)

The Phenstatin acryl ate was then polymerized with NIPAAm. The following procedure is for a polymer containing 5 mol % acrylate and 95 mol % NIPAAm. 5 mL of Tetrahydrofuiran (THF) is then added to the RBF. Next, 86 mg of acrylate, 5.35 mg of AlBN and 0.5 g of NIPAAm are added to the THF. The mixture was then allowed to polymerize at 60° C. overnight while stirring. The reaction was covered with aluminum foil during polymerization to reduce light. Once the reaction was complete, it was dropped into 50 mL of diethyl ether. The precipitate was then collected by vacuum filtration. The polymer was further dried in a desiccator under vacuum.

FTIR

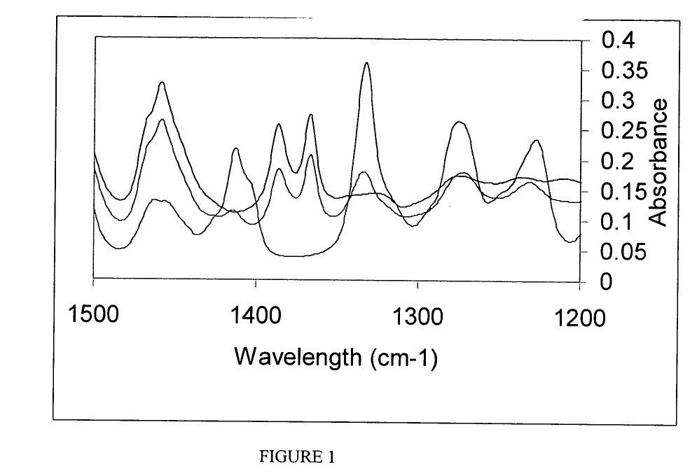

The successful synthesis of acrylated phenstatin was and polymerization of poly(NIPAAm-co-phenstatin) was confirmed using FTIR. FIG. 1 shows FTIR for the acrylated phenstatin and for the poly(NIPAAM-co-phenstatin) compared with poly(NIPAAm). A peak in the acrylated phenstatin at approximately 1750 cm−1 confirms the addition of the acrylate to phenstatin. This peak at 1750 cm−1 for the acrylate phenstatin (C═O associated with the Acrylic group) disappears in the polymer. This indicates incorporation in the polymer. Distinguishing peaks found in acrylated phenstatin but not in poly(N-isopropylacrylamide) at 1416 cm−1 and 1334 cm−1 were also found in the poly(N-isopropylacrylamide-co-Phenstatin).

NMR

Confirmation of the correct synthesis of acrylated Phenstatin was achieved using NMR. The peaks around 4 ppm are the methoxy protons. The vinyl protons appear between 6 and 7 ppm. The remaining signals between 7 and 8 ppm are the benzylic protons.

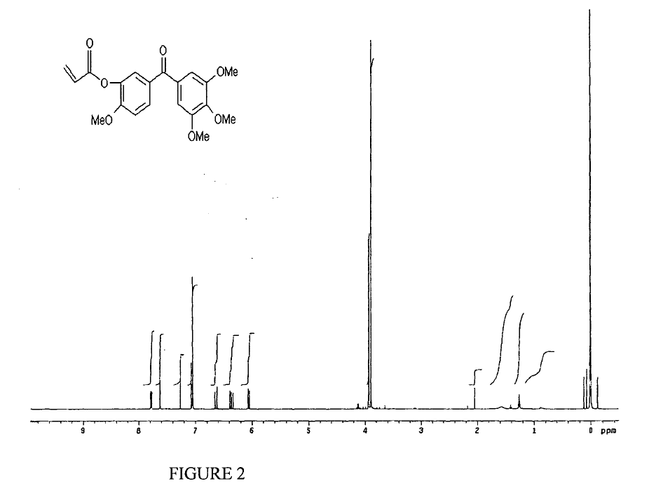

FIG. 2 illustrates the proton NMR of Acrylated Phenstatin.

### Example 4

This example illustrates the preparation of NIPPAn/Phenstatin with a carbonate linkage.

The carbonate bond is less stable and thus provides faster release of the antineoplastic agent. The reaction scheme is summarized in the following diagram. In the top reaction, Isovanillin, a model for Phenstatin, is converted to an acrylate by reacting it with Isopropenyl Chloroformate. The bottom reaction depicts the polymerization of Isovanillin Acrylate with NIPAAm.

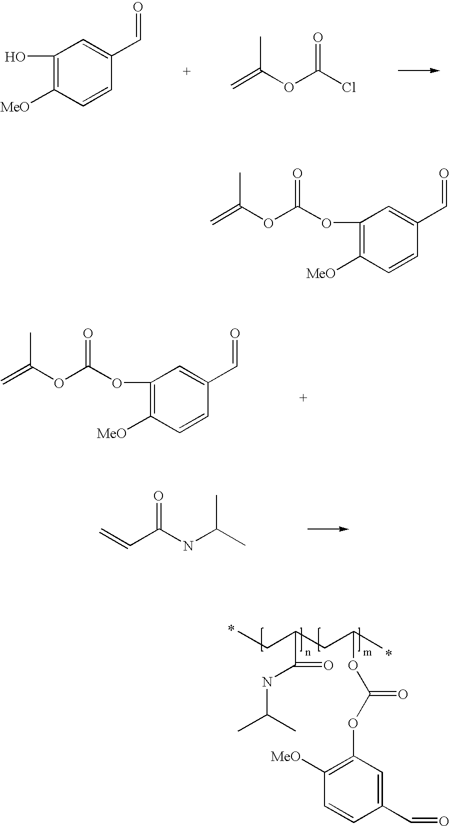

An acrylic group was added to Phenstatin by reacting Phenstatin with Isopropenyl Chloroformate (IPCF)

Acrylation of Pheristatin with a Carbonate Linkage

Before starting the acrylation, all glassware (Round Bottom Flask (RBF), Dropper Funnel, Glass funnel, graduated cylinder) must be dried in an oven overnight. Once the glassware is dry, it is then placed on a condenser and nitrogen gas is sent through the system as it cools. After the glassware is set up and dried, 10 mL of anhydrous dichioromethane (DCM) is measured into the RBF. Next, 100 mg of Phenstatin is measured into the RBF. A stir bar is then added to stir the mixture. After the Phenstatin is added, 98 μL of triethylamine is pipetted into the RBF.

Once all the chemicals are added to the RBF, the mixture is stirred until it dissolved. When the mixture dissolves, it is then placed on an ice bath. The dropper funnel is then closed and 1 mL of DCM is added. After the DCM is added, 130 μL of Isopropenyl Chloroformate (IPCF) is carefully added to the DCM in the dropper funnel. The IPCF and DCM are allowed to mix thoroughly. Once they are mixed, the DCM/IPCF mixture is slowly dropped into the RBF over the ice bath. After all the solution is dropped into the RBF, the dropper funnel is closed and the reaction is allowed to proceed for 24 hours.

The acrylate was then collected by taking the reaction off of the ice bath. The mixture was filtered to remove the triethylamine salt formed as a byproduct. Next, the reaction mixture was added to a separatory funnel. Four passes of 1 N HCl, volume of 15 mL each, were then used to extract the remaining triethylamine. The reaction mixture was then dropped into 150 mL of hexane while being stirred. This was then filtered. Once the mixture had been filtered, its volume was reduced by using a rotary evaporator. The volume was reduced from 150 mL to about 50 mL. Once its volume was reduced to 50 mL, the hexane was placed on an ice bath so that the contents could recrystallize. The hexane was then filtered by vacuum filtration and the product was collected. The product was placed into a small tared scintillation vial and it was dried in a dessicator under vacuum.

Polymerization of Phenstatin Acrylate (5 mol % Acrylate) using a Carbonate Linkage

Polymerization of the Phenstatin Acrylate was performed as given in Example 1 or 2.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

References

1. Pettit, G. R. et al. Antineoplastic Agents: 443 Synthesis of the cancer cell growth inhibitor hydroxyphenstatin and its sodium diphosphate prodrug”, Journal of Medicinal Chemistry, 2000, 43(14); p. 2731-2737.

2. Pettit, G. R. et al. Antineoplastic agents 379 Synthesis of Phenstatin Phosphate Journal of Medicinal Chemistry, 1998 41 (10) p. 1688-1695.

3. Epstein, A. H. et al., Intravenous delivery of5 ′-iododeocyuridine during hyperfractionated radiotherapy for locally advanced head and neck cancers: Results of a pilot study. Laryngoscope, 1998; 108(7) p. 1090-1094.

4. Koeller, J. M. et al., Pharmaceutial Issues of Paclitaxel. Annals of Pharmacotherapy, 1994, 28(5) p. S5-S36.

5. Zhang, X. Z. et al. A novel thermo-responsive drug delivery system with positive controlled release International Journal of Pharmaceutics, 2002, 235(12), p. 43-50.

6. Chiantore, O. M. et al. Solution properties of poly(N-isopropylacrylamide) Makromol. Chem. 1979, 180 p. 969-973.

1. A drug delivery system for localized delivery of Phenstatin to a tumor in vivo comprising the polymer poly(N-isopropylacrylamide) chemically bound to Phenstatin.

2. The drug delivery system of claim 1 having the formula poly(N-isopropylacrylamide-co-phenstatin).

3. The drug delivery system of claim 1 having a lower critical solution temperature above 25° C. and below body temperature.

4. The drug delivery system of claim 1 containing about 5 mol % Phenastin acrylate.

5. The drug delivery system of claim 1 comprising in addition AAc co-polymerized with poly(N-isopropylacrylamide).

6. The drug delivery system of claim 5 containing about 5 mol % to 10 mol % Phenstin acrylate.

7. The drug delivery system of claim 1 wherein Phenstatin acrylate is bound to poly(N-isopropylacrylamide) through an ester bond.

8. The drug delivery system of claim 7 wherein Phenstatin acrylate is bound to poly(N-isopropylacrylamide)through a carbonate bond.

9. A method of preparing the compound of claim 1 comprising the steps of
i. preparing Phenstatin acrylate; and
ii. polymerizing said Phenstatin acrylate and poly(N-isopropylacrylamide).

10. The method of claim 9 wherein said polymerizing step comprises co-polymerization with acrylic acid.

11. The method of claim 9 wherein Phenstatin acrylate is prepared by reaction of Phenstatin with acryloyl chloride.

12. The method of claim 9 wherein Phenstatin acrylate is prepared by reaction of Phenstatin with isopropenyl chloroformate.

13. A method of treating a cancerous tumor wherein the drug delivery system of claim 1 is locally injected into tumor-containing tissue.

14. The method of claim 13 wherein said tumor tissue is a breast, prostate, lung or bowel tissue.
